(19) United States
(12) Patent Application Publication
(10) Pub. No.: US 2009/0215073 A1
(43) Pub. Date: Aug. 27, 2009

(54) Layered peptide/antigen arrays - for high-throughput antibody screening of clinical samples

(76) Inventors: Michael Emmert-Buck, Easton, MD (US); Gallya Gannot, Rockville, MD (US)

Correspondence Address: Jonathan E. Grant, 2107 Hounds Run Place, Silver Spring, MD, 20906, US

(21) Appl. No.: 12/289,736

(22) Filed: Nov. 3, 2008

Related U.S. Application Data
(63) Continuation of application No. 11/189,038, filed on Jul. 26, 2005, now abandoned.
Application No. 11/189,038 is a continuation-in-part of application No. 10/048,194, filed on Feb. 15, 2002, now Pat. No. 7,214,477.
(60) Provisional application No. 60/591,749, filed on Jul. 27, 2004.

(51) Int. Cl. G01N 33/53

(52) U.S. Cl. 435/7.1

(57) ABSTRACT

A method and composition for the identification of biomolecule in a sample are disclosed. The method comprises obtaining a coated capture membrane stack comprising a plurality of capture membranes with each capture membrane coated with a different peptide. The membrane stack is exposed to a sample, and, after a given amount of time for the sample to permeate the membrane stack, the membrane stack is removed from the sample carrier and the capture membrane to which the biomolecule adheres is identified.

This application is a CIP of pending U.S. application Ser. No. 10/048,194, filed Feb. 15, 2002, which is based on PCT/00/20354, filed Jul. 26, 2000. This application also claims priority of Provisional Application Ser. No. 60/591,749, filed Jul. 27, 2004. The subject matter of the aforesaid applications are incorporated by reference in their entireties.

### STATEMENT OF GOVERNMENT RIGHTS

At least one of the inventors is an employee of an agency of the Government of the United States, and the government may have certain rights in this invention.

### FIELD OF THE DISCLOSURE

The present disclosure is directed to compositions and methods for identifying biomolecules in a biological sample.

### BACKGROUND

Antibodies play a major role in the adaptive immune response due to high-affinity binding to specific epitopes on target antigens. Indeed, human sera contain approximately 10 million different antibodies with activity against a wide-range of potential pathogens. In clinical medicine, sera from patients are frequently analyzed for the presence or absence of a few specific antibodies as a guide to diagnosis and therapy. In fact, many if not most infectious or auto-immune diseases are diagnosed by testing for the presence of these antibodies.

Over the years, there have been many efforts to develop an improved and faster method of isolating a biomolecule, preferably an antibody or antigen, with the goal of diagnosing a condition or illness.

Numerous devices have involved using immuno-chromatographic techniques. For example, U.S. Pat. Nos. 6,060,326 and 5,945,294 (Frank et al.) disclose methods to detect canine IgE using a canine Fc epsilon receptor to detect canine IgE antibodies in a biological sample from a canine.

Other detection methods rely on traditional techniques for identification of certain antibodies or diseases.

U.S. Pat. No. 5,200,344 (Blaser et al) uses a purified p28kd protein from H. pylori to detect IgA, IgM and IgG antibody in ELISA and Western Blot tests. The test(s) require a conjugate and enzyme substrate and two wash steps to detect the antibody. The antigenic compositions of the invention are contacted with samples such as body fluids suspected of containing C. coli- or C. jejuni-specific antibodies. Following such contacting, known methods are used to determine the extent of formation of an antigen/antibody complex comprised of immunoglobulin bound to antigens from the antigenic composition of the invention. When formation of the complex exceeds a predetermined positive threshold value, the test is positive for presence of C. jejuni or C. coli-specific antibody.

U.S. Pat. No. 6,068,985 (Cripps) discloses a method which uses saliva to detect IgG in both the Western Blot and ELISA tests. This detection method requires the use of an enzyme conjugate and enzyme substrate and two wash steps to detect the antibody.

While these patents do describe methods or assays for testing certain biomolecules, they all have the limitation of only being able to test, at most, a few targets at a time. Their ability to screen for specific diseases is limited to only those antibodies or ligands positioned on test strips or in wells. Most of these tests involve an antibody-ligand or “antibody-ligand-nonhuman-antibody” sandwich reactions.

An emerging approach for autoantibody detection involves the use of protein microarrays comprising a grid of many different peptides printed, spotted, or synthesized on the surface of glass slides or other planar surfaces. For example, one group spotted tumor derived protein onto coated microscope slides that were then hybridized with individual sera from prostate cancer patients and healthy subjects to profile autoantibodies in the samples. U.S. Pat. No. 6,815,078 discloses a gelatin-based substrate for fabricating protein arrays, the substrate comprising: gelatin having at least one surface; a polymer scaffold affixed to the gelatin surface; wherein the polymer in the scaffold is rich in reactive units capable of immobilizing proteins.

However, these microarrays have the disadvantage of only being able to test one sample at a time.

It would be desirable to have a tool that permits multiple patient samples to be tested in parallel, each against multiple antibodies. This would be particularly advantageous in the case of bioterrorism defense when one if found with an immediate need to quickly screen an entire population for multiple infectious diseases in parallel.

### SUMMARY OF THE DISCLOSURE

In this disclosure, high throughput methods are used to detect and quantify antibodies in sera and other patient specimens. These methods have utility for many clinical and laboratory studies, including those associated with cancer detection, microbial exposures, and auto-immune diseases. The high throughput methods and related kits and compositions answer the need for high throughput detection of antibodies in biological samples, such as serum, and allows many different samples to be tested simultaneously for many different antibodies, quickly and inexpensively.

In one embodiment of the disclosure, a plurality of membranes are each coated with a peptide or antigen, with each peptide or antigen having an affinity to a particular antibody or biomolecule (herein designated as direct LPA). These membranes are placed one on top of the other to form a membrane stack. Test samples, preferably sera or saliva, are arranged in a multi-well grid or plate. The membrane stack is placed in contact with the grid or plate, and the samples travel through the membrane layers while maintaining their two-dimensional locations within the network. If present in a sample, antibodies are specifically captured by their target peptide as they pass through the membranes, and are subsequently detected using standard secondary antibody-based methods.

The invention may be described in terms of the x-y plane (dimension) of the biological sample platform (e.g., a multiwell plate), and the z-dimension representing the stack of layered capture membranes. Such an arrangement allows for the testing or profiling of numerous samples at one time. A single test can easily be performed for 50-100 or more antibodies, which will produce thousands of measurements in minutes.

There are advantages to detecting an antibody with a protein coated membrane. Antibodies readily and easily migrate and naturally migrate in the body to proteins. Peptides have a longer shelf life than antibodies. Additionally, it is harder to coat antibodies on the membranes. In contrast, no orientation problems arise when coating a protein on a membrane. Also, it is much cheaper to coat a membrane with a protein than with antibodies. By testing for antibodies in bodily fluids (e.g., serum, saliva, tears, spinal fluid, urine, sweat, etc.) they and related small biomolecules easily pass in a fluid up through the membranes. By coating an entire surface with the specific target peptide, the specific antibody being tested will more readily bind the intended target. Accuracy is thus assured. As will be seen by the histographs in the figures, imagery is sharp and distinct with the present invention.

In one embodiment of the disclosure, a Layered Peptide Array (LPA) serves as a screening tool by detecting antibodies in a highly multiplexed format. As part of the disclosure, a prototype LPA is capable of producing approximately 5,000 measurements per experiment, and appears to be scalable to higher throughput levels.

In another embodiment of the disclosure, tests can be performed for a number of autoimmune diseases, including but not limited to Sjögren's syndrome.

In yet another embodiment of the disclosure, the LPA system and method may be used to screen for numerous autoimmune diseases at one time.

In another embodiment of the disclosure, it is proposed that auto-antibodies recognizing tumor antigens can serve as effective screening tools for cancer.

In another embodiment, patient sera could be tested for the presence of any one of a relatively large panel of antibodies against unique antigens expressed by neoplastic cells. Applied successfully, physicians can screen whole populations (or specific at-risk populations) for the presence or recurrence of a tumor as an adjunctive tool to current diagnostic techniques.

In another embodiment of the disclosure, sera samples can be screened for a panel of antibodies directed against toxic or infectious agents.

In yet another embodiment of the disclosure, tissues and samples may be examined by an indirect LPA system, whereupon antibodies are prebound to target antigens on a tissue section or other solid surface.

The proposed methods and compositions will allow for multiplex antibody screening which will facilitate research efforts, allowing investigators to rapidly and inexpensively identify hybridoma clones that produce antibodies with a well-characterized antigen binding profile.

Before proceeding further, a definition of the terms used and their applicability to the disclosure is needed.

“Biomolecules” are molecules typically produced by living organisms. These molecules may include peptides, proteins, glycoproteins, nucleic acids, fatty acids, and carbohydrates and antibodies.

“Target biomolecule” is a biomolecule that one seeks to identify, analyze, or measure in a sample that has an affinity for the captor molecule.

“Sample” means a material that contains biomolecules. A sample in this case may typically include tissue, gels, bodily fluids, and individual cells in suspensions or in pellet form, as well as materials in containers of biomolecules such as microtiter plates.

“Captor” or “Capture molecule” is a peptide or antigen that is anchored to a membrane and has an affinity (such as a selective affinity) for one of the biomolecules in the sample (e.g. antibody).

“Conjugate” means to chemically bond two or more compounds.

“Affinity” means the chemical attraction or force between molecules.

“Capacity” means the ability to receive, hold, or absorb biomolecules from the sample.

“Detector” means a molecule, such as an antibody, antigen, or DNA probe that is free in solution (e.g., not anchored to a membrane), and has an affinity for one of the sample components.

“Antibody” refers to polyclonal, monoclonal, or chimeric antibodies and includes any portion of an antibody or a fragment of an antibody, such as a Fab fragment, as long as it is capable of binding to an antigen. The antibody, or portion thereof, may be purified, recombinant, or synthetic.

“Membrane” or “Substrate” means a thin sheet of natural or synthetic material that is porous or otherwise at least partially permeable to biomolecules.

“Stack” refers to adjacent membranes, whether stacked horizontally, vertically, at an angle, or in some other direction. The substrates may be spaced or touching, AND may be contiguous.

“Bispecific antibody conjugate” means a molecule produced by conjugation of one antibody that is qualified for immunohistochemistry with one antibody that is qualified for immunoblotting. A bivalent cross-linker chemical, such as SPDP or such as a disulfide bond, can be used to link the two antibodies, such as with a disulfide bond.

“Peptides” are a composed of acid units (amino acids) chemically bound together with amide linkages (CONH) with elimination of water. Peptides can be as few as two or three units in length, or up to twenty or more units.

“Antigen” means any material that elicits production of, or is specifically bound by, one or more antibodies.

“Recognition” means the chemical bonding of one molecule with another specific molecule.

“Primary antibody” means an antibody which recognizes specific biomolecular content in the sample, and is conjugated with a shuttle antibody to form a bispecific antibody conjugate.

“Shuttle antibody” means an antibody or portion thereof that selectively recognizes specific peptides, and may be conjugated with a primary antibody to form a bispecific antibody conjugate.

With the foregoing and other objects, advantages and features of the disclosure that will become hereinafter apparent, the nature of the invention may be more clearly understood by reference to the following detailed description of the invention, the appended claims and to the several views illustrated in the drawings.

### BRIEF DESCRIPTION OF THE DRAWINGS

FIG. 1 is a schematic illustration of direct antibody capture by the LPA system;

FIG. 2 is a schematic of indirect antibody detection;

FIG. 3 is a histograph of the LPA Prototype antibody control experiment;

FIG. 4 is a LPA prototype of the serum samples;

FIG. 5 is a graph of the test results of the serum;

FIG. 6 is a graph of the comparisons between the LPA system and the standard ELISA system.

FIG. 7 is a graph of the comparison of the intensity of the signals between the LPA system and the standard ELISA system;

FIG. 8 is an exploded view of the membrane stack, wells and vacuum system principal component analysis (PCS) clustering for patients and controls;

FIG. 9 shows the histographs of the sample and controls, along with the dilution rate of the samples;

FIG. 10 is a histograph of the results of an LPA showing the effects of sample dilution on the system;

FIG. 11 is graph of the results of an LPA showing the intensity of the signal of sample dilution on the system;

FIG. 12 is a comparison of the results between LPA Prototype #2 and a standard ELISA test of various serum samples;

FIG. 13 is a graph showing the quantitative signal measurements between the LPA and ELISA tests;

FIG. 14 is a graph showing a comparison of antibodies in serum and saliva for patients and normal volunteers;

FIG. 15 lists the ANOVA values of saliva and serum for patents and normal volunteers;

FIG. 16 is a principal component analysis for the clustering of patients and controls;

FIG. 17 shows the annotated areas on an enhanced signal of an iLPA whole mount prostate section;

FIG. 18 shows the signal on membranes coated with PSA peptide and membranes coated with non-relevant peptide in an iLPA of a whole mount prostate section;

FIG. 19 are histographs of SS minor salivary glands using an iLPA test

FIG. 20 is a chart of the intensity of the signals for the SS minor salivary glands using and iLPA test;

FIG. 21 is a graph summarizing the iLPA results for minor glands;

FIG. 22 is a wire mash of data points according to iLPA/IHC analysis;

FIG. 23 is a comparison of iLPA and IHC for 10 prostate whole mount frozen cases; and

FIG. 24 is a formalin fixed and paraffin embedded tissue arrays using iLPA technology.

### DETAILED DESCRIPTION OF THE DISCLOSURE

With reference to FIGS. 1 and 7, the invention generally comprises a plurality (or stack) 12 of coated membranes. In one preferred embodiment, the coating of each membrane is a peptide, polypeptide, or a protein, which is specific for a specific antibody or some specific biomolecule. The number of membranes 14 in stack 12 may be as few as two or as many as 100. The number of membranes in a stack depends largely on the number of targets sought to be identified in the sample. Each membrane is coated with a different peptide or antigen containing an active epitope for the antibody of interest.

The membranes 14 are preferably constructed of a thin porous substrate that is coated with peptides or antigens. The substrate is preferably constructed of polycarbonate or a similar polymeric material that maintains sufficient structural integrity despite being made porous and very thin. In lieu of polycarbonate this material may include, for example, polyester or polyethylene terephthalate. The membrane may also be comprised of a cellulose derivative such as cellulose acetate, polyolefins, (e.g., polyethylene, polypropylene, etc.), gels, or other porous materials.

In one aspect of the invention, the substrates may be “track-etched membranes” (a/k/a “screen membranes”). These membranes are formed by a process that creates well-defined pores by exposing a dense film to ionizing radiation forming damage tracks. This is followed by etching of the damaged tracks into pores by a strong alkaline solution. A description of this process may be found on the Internet site of General Electric Water Technologies (Trevose, Pa.) at http://www.gewater.com/library/tp/322_Basic_Principles.jsp under the heading, “Basic Principles of Microfiltration” (herein incorporated by reference). Examples of membranes that may be employed as the substrate include the Isopore™ (polycarbonate film) membrane available from Millipore (Billerica, Mass.), the Poretics® Polycarbonate or Polyester membranes available from GE Osmonics Labstore (Trevose, Pa.), or the Cyclopore™ Polycarbonate or Polyester membranes available from Whatman (Clifton, N.J.).

In lieu of a track-etched membrane, a depth or tortuous pore membrane might be employed if its capacity can be rendered low enough to permit a stack of three of more such membranes to be used in the present invention. This could be accomplished, for, by example, casting the membrane very thin, far thinner than the thickness of depth membranes conventionally employed (150 microns). Alternatively, blocking certain binding sites could lower the capacity of conventional depth membranes.

Each membrane substrate is coated on one or both sides with a peptide or other antigen so that it may specifically bind a target such as an antibody. Peptides may be obtained from a variety of commercial sources or custom made by the user. For example, recombinant DNA technologies, peptide synthesis, or other techniques may be used by of those skilled in the art.

The membrane substrates, after being cut to the desired size depending on the size of the sample being tested, are incubated in a solution made up of the peptide or antigen that is diluted in a suitable buffer such as TRIS or phosphate buffered saline (PBS). The ratio of peptide to buffer may be 1:10 to 1:100 but is preferably 1:50 (see table 1). The time of incubation may be between about 3 and about 24 hours but is preferably incubated about 4 hours. In brief, uncoated membranes are incubated in a solution that contains peptides (such as those shown in Table I) or the antigen (such as those shown in Table I) diluted in PBS. Every membrane is incubated with at least one peptide (or other antigen) and thus can bind one antibody that is specific for that membrane. Alternatively, the membrane may be with two-different peptides, and two antibodies could then bind to the membrane. The membranes are incubated with the peptides/antigens for 4 hours at room temperature with shaking. Membranes are washed in a TBST solution ((1) IM Tris Hcl pH 8.0-50 ml (50 mM); (2) 5 M NaCl-30 ml (150 mM); (3) Tween20 0.5 ml (p150 mM) in a total volume of 1 liter ddH₂O) three times for 5 minutes and applied to the LPA system.

It should be appreciated by those skilled in the art that alternative coating methods, such as spray coating and lamination, may be employed. Multiple coatings might be employed to ensure that the entire membrane substrate is coated. In lieu of coating, the peptide or antigen shape may be imprinted or cast into the membrane substrate when it is manufactured using techniques similar to those described by Henricksen et al. (“Artificial Antibodies,” IVD Technology Magazine, July 1996, incorporated herein by reference) thereby providing an uncoated membrane having the same or similar binding affinity of coated membranes 14. It should be understood that in some cases, only the surface that faces the sample need be coated. It should also be appreciated that some substrates employed (e.g., polyester) may not require a coating in order to have an affinity for certain biomolecules.

In order to provide structural rigidity to membranes so that they may be separated from one another and individually processed, frames 22 (FIG. 7) may be optionally mounted around the membranes. Frames may compromise a generally “U” shaped configuration covering three sides of the membranes while leaving one side open to permit the manual removal of air pockets. Alternatively, frames may cover all four sides of the membrane. Frames may be mounted to the perimeter of membranes by various means, including use of adhesives such as rubber cement, or 3M adhesive, various heat-sealing techniques, or sonic welding. The frames might also be treated with an agent to block or prevent the proteins or nucleic acids from binding to the frame.

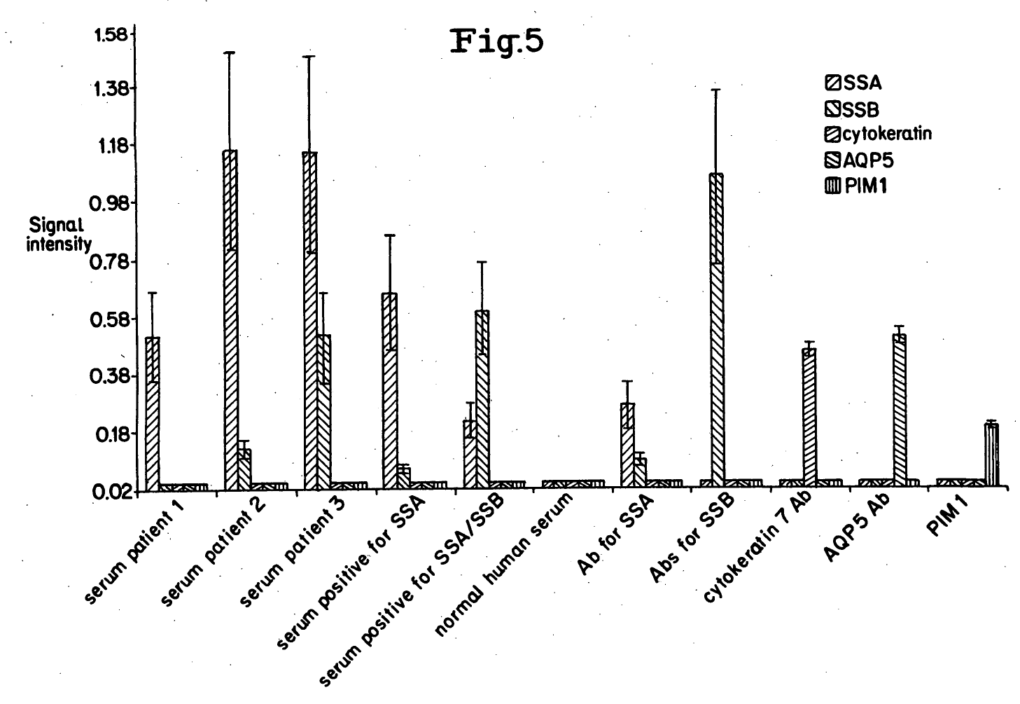

In FIG. 1(a) and 7, test samples are arranged in a multi-well grid 16 (FIG. 1a) or wells 24 of the vacuum transfer manifold of FIG. 5. It is preferred that there be a different test sample in each well, although at least one well may contain a positive control. Alternatively, there may be negative control, or there may be both a positive control and a negative control, with the positive control containing antibodies that will be specific for each of the peptides coating one of the membranes. The negative control can be a random assortment of antibodies that should be specific for none of the peptides.

The stack of membranes 12 is placed in contact with the wells. A blotting sheet (not shown) may be placed on top of the membrane stack 14. If present in a sample, antibodies are specifically captured by their target peptide as they pass through the layers, and subsequently detected using standard secondary antibody-based methods. More specifically, antibodies move from the multi-well grid and through the membranes via capillary action (or vacuum force), where they are captured by/react with their corresponding epitopes. As the identity of the samples along the x-y plane of the biological sample platform (the array of samples in a multiwell plate or tissue section) are known, and the peptides or proteins on the z-dimension representing the stack of capture membranes 14 are also known, determination of the positive sample is quick and easy, even without the use of a computer. With this approach (that may be referred to as a “layered peptide array” or “LPA”), multiple samples can be profiled or analyzed for multiple targets producing thousands of measurements in minutes.

In addition to the liquids listed above, tissue and cellular samples can also be tested for the presence of antibodies. Advantageously, when the samples are oriented in a two dimensional format such as with a standard multiwell (96, 384, or 1536 well) plates, 2-way multiplex analysis is enabled, wherein multiple samples may be simultaneously screened against multiple targets in parallel.

In a variation to the method given for use of the coated membranes, a membrane stack 12 may be used in conjunction with a multiwell vacuum transfer apparatus 20 (FIG. 7), with the vacuum 25 positioned underneath the membranes. As set forth herein, in lieu of a multiwell plate, membranes 14 may capture antibodies from samples embedded in agarose gels. The vacuum

### Serum Profiling—Direct LPA

One useful application of the present invention is the profiling of autoantibodies in serum samples in a multiwell vacuum transfer manifold. More specifically, multiple patient samples to be applied to the wells 24 and screened for the presence of multiple autoantibodies on each of membrane layers 14. With reference to FIG. 7, coated membranes 14 are placed within a multiwell vacuum transfer manifold 20 such as the one described in co-pending PCT International Patent Application PCT/US01/44009.

Although manifold 20 has 96 wells, multiwell plate manifolds with 384 and 1536 wells are commercially available from a variety of sources and may also be employed. Samples comprising bodily fluids (e.g., serum, saliva, tears, spinal fluid, urine) are diluted in TBS in a concentration of about 1:50-1:200.

Using a micropipette instrument, the diluted samples are applied to some or all of the wells, thereby permitting multiple patient samples to be screened in parallel. Additionally, positive control samples can be added to some of the wells. For example,—the membranes may be coated with two peptides (or two antigens or a peptide and antigen). Antibodies specific for the coated peptides/antigens will react on the membrane and be detected using two different secondary antibody-detection system. This procedure will enable the use of a control antibody-peptide pair for normalization of the signal.

With the diluted samples in place, vacuum pressure is applied for between about 2 and about 20 minutes, preferably about 5 minutes, although the duration of vacuum transfer may vary depending on the sample and the vacuum system. Following transfer, the membranes are removed from the manifold and washed in a suitable buffer such as TBST. The membranes are then incubated in a labeled secondary antibody such as (goat) anti human FITC or cy5 conjugated antibody, available from a variety of commercial sources, such as Santa Cruz Biotechnology. Unbound secondary antibody is washed off each of the membranes with buffer. Detection of binding is accomplished using FITC or cy5 filter in a laser scanner (such as Typhoon scanner Typhoon 9410, Amersham Biosciences, NJ, USA). Alternatively, the membrane stack can be left inside the manifold and washed and incubated in the secondary antibody within the device.

The sample may be a liquid, such as plasma, serum, or saliva, or tissue, and may be applied to the membranes via each well of a multi-well plate. Each membrane in the stack has been coated with a different substrate that will selectively bind different specific biomolecular content eluted from the sample. The membrane stack may consist of between about 5 and about 50 membranes but 100 or more membranes may also be employed. After the elution, the membranes may be separated, and further treated with a detector biomolecule, such as a tagged antibody, that will detect the presence of the bound biomolecular content so that it may be visualized on the membrane by a laser scanning device or CCD camera. Membrane images can be imported to an image analysis program such as ImagePro 4.5 analysis software (MediaCybernetics, MD, USA) whereupon the optical density can be calculated for each sample. Digitalized images may be analyzed using any of a number of image analysis software packages.

A key application involves the use of membrane stacks 12 with multiwell plate 20 to generate antibody profiles from multiple serum samples in parallel. There are great efforts underway to improve early disease detection, especially for cancer, which is most effectively treated in its earliest stages. One emerging paradigm for early detection of cancer has centered on early detection of autoantibodies to the cancer. For example, anti-malignin antibody (most effectively using the AMAS—anti-malignin antibody in serum-test) is indicative of breast cancer (Thomthwaite, Cancer Letters 148:39, 2000). Autoantibodies against the complete p53 protein and 18-mer peptides of p53, such as IgG1 IgM, and IgM plus IgG2, are indicative of ovarian cancer (Vennegoor, Cancer Letters 115:93, 1997, incorporated herein by reference). Presence of p53 antibodies can also be an early indicator of colorectal cancer (Broll et al., Colorectal Disease 16:22, 2001, incorporated herein by reference).

With urological cancers—including prostatic, transitional cell carcinoma of the urinary tract, and renal cell cancer,—p53 autoantibody expression “seems to be a late but significant event in urological tumor development” (Lang et al., British J Urology 82:721, 1998; see also Wunderlich, Urologia Internationalis 64:13, 2000, incorporated herein by reference).

Numerous other cancers may be tested, given the galaxy of anticancer antibodies that are created in the presence of the various cancers that (can) form in the body.

Novel insights into pathobiology, analysis of multiplex auto-antibody data sets likely will provide value beyond the information provided by standard single analyte tests. For example, Zhang et al studied antibodies in cancer patient sera and found that examining a profile of seven different autoantibodies raised the cancer detection rate significantly compared to analyzing a single antibody (See “Enhancement of antibody detection in cancer using panel of recombinant tumor associated antigens.” Cancer Epidemiology Biomarkers Prev. 2003 12(2), p. 136-43, incorporated herein by reference).

Early autoantibody detection, however, is not limited to cancer detection. It is also useful in diagnosing such autoimmune diseases as rheumatoid arthritis and primary biliary cirrhosis. Elevated levels of IgM-RF (IgM rheumatoid factor) or anti-CCP (anti-cyclic citrullinated peptide) imply a high risk of rheumatoid arthritis development (Nielen et al., Arthritis & Rheumatism 50:380, 2004, incorporated herein by reference). Moreover, in patients already diagnosed with rheumatoid arthritis, autoantibodies against GPI (glucose-6-phosphate isomerase) are associated with extraarticular complications (van Gaalen et al., Arthritis & Rheumatism 50:395, 2004, incorporated herein by reference).

High titer autoantibodies against certain mitochondrial antigens are associated with primary biliary cirrhosis (Mattalia et al., J. Autoimmunity 10:491, 1997, incorporated herein by reference). Other diseases associated with autoantibodies to mitotic spindle apparatus include Sjögren's syndrome, Raynaud's phenomenon, systematic sclerosis, undifferentiated connective tissue disease, polymyositis, polymyalgia rheumatica, systemic and discoid lupus, vitiglio, epidermolysis bullosa acquisita, melanoma, dilated cardiomyopathy, mycoplasma, Hashimoto's thyroiditis, osteoarthritis arthralgia, and sciatica (Shoenfeld et. al., Autoantigens and Autoantibodies: Diagnostic Tools and Clues to Understanding Autoimmunity, RHEUMA21ST, 2000, incorporated herein by reference). Other autoimmune diseases that could be tested include but are not limited to: sclerodomea, Goodpasture's syndrome, Wegener's granulomastosis, temporal arterosis, and pemphigus.

The disclosed methods could also be used to detect other localized autoimmune diseases such as diabetes mellitus, celiac disease(s) (which includes Crohn's disease, ulcerative colitis, Addision's disease, primary biliary sclerosis, sclerosing cholangitis, and autoimmune hepatitis.

This method can be used not only to test for autoimmune diseases but to test for exposure to pathogens. Pathogens that could be tested for include virtually the entire array of pathogenic or invasive bacteria, viruses, molds, and funguses.

Similarly, this technique can also be used to test for certain chemical and metal pathogens, including but not limited to bacterial food toxins, environmental toxins, etc that generate an immune response.

An aspect of the present invention relates to the detection of antibodies in a sample. The antibody may be polyclonal, monoclonal, or chimerical, or be any portion of an antibody, such as a Fib fragment, as long as it, is capable of binding to a peptide coated on a membrane. The portion of the antibody may be made by fragmenting the antibody, or the portion may be produced recombinant or synthetically. All of these approaches are well-known in the art. Whole antibodies and polyclonal antibodies may be present in, for example, sera collected from test subjects. Antibody fragments or portions may be generated for in vitro characterization in, for example, high through-put screening.

Stack 12 may comprise membrane 14 specific to entirely different types of targets. For example, one membrane layer may capture antibodies to an autoimmune disease while an adjacent layer may capture antibodies associated with cancer. In another aspect of the invention, other proteins (as well as antibodies) may be captured—such as interleukins or interferon's involved in disease states. In these instances, some membranes may be coated with antigens, and others coated with antibodies or portions thereof. Hence, different types of target biomolecules may be detected in different layers. For example, both antibodies and other proteins may be detected in parallel by applying different antigens or antibodies to different layers of the membrane stack.

Controls and screening of the type of coating on each membrane will prevent unwanted interactions resulting in false positives.

However, tests can also be performed wherein the proteins of the membrane will bind to specific proteins in solution of the sample. Such protein-protein interactions can be found, for example, with certain bacterial and other biological toxins.

Another aspect of the present disclosure is providing a kit that includes a group of membranes in a stack or other configuration that permits them to be stacked, and different detectors.

Another object of the present disclosure answers the need for high throughput detection of antibodies in biological samples, such as plasma or tissue. For example, many different samples can be tested simultaneously (as well as quickly and inexpensively) for the presence of one or more antibodies. This is an improvement over the Enzyme-Linked Immunosorbent Assays (“ELISAs”), which only screen for one antigen per sample. For example, the present invention provides for technology in which a patient may be tested for several different antibodies, the presence of which may allow for the diagnosis of a patient who has specific maladies. Moreover, many different patients may be tested for such antibodies at the same time, providing easy and invaluable plasma or tissue comparisons. The antibodies chosen for possible detection may, for example, be those produced in the early stages of rheumatoid arthritis and certain cancers, such as breast, ovarian, bladder, colorectal, or urological cancer.

Another embodiment of the LPA system provides for a method for eluting the biomolecules from the sample, and visualizing them on membranes having a special affinity for the biomolecules of interest with the biomolecule preferably being antibodies. The membranes, in a stacked or layered configuration, are brought into contact with the sample and reagents, and reaction conditions are provided so that the biomolecules are eluted from the sample onto the membranes, whereupon the biomolecules can be visualized using a variety of techniques, as set forth herein.

The material of the membranes may maintain a relative relationship of biomolecules as they move through the membranes, so that the same biomolecule (or group of biomolecules) move through the plurality of membranes at corresponding positions.

A direct capture example of the invention includes a method of detecting an analyte in a biological sample using stacked contiguous layered membranes that permit biomolecules to move through a plurality of the membranes, while directly capturing the biomolecules on one or more of the membranes. Biomolecules from the sample move through the membranes under conditions that allow one or more of the membranes to directly capture the biomolecules. Biomolecules of interest are concurrently or subsequently detected on the membranes by virtue of their presence on a membrane with a selective affinity.

### Tissue Profiling/Indirect LPA

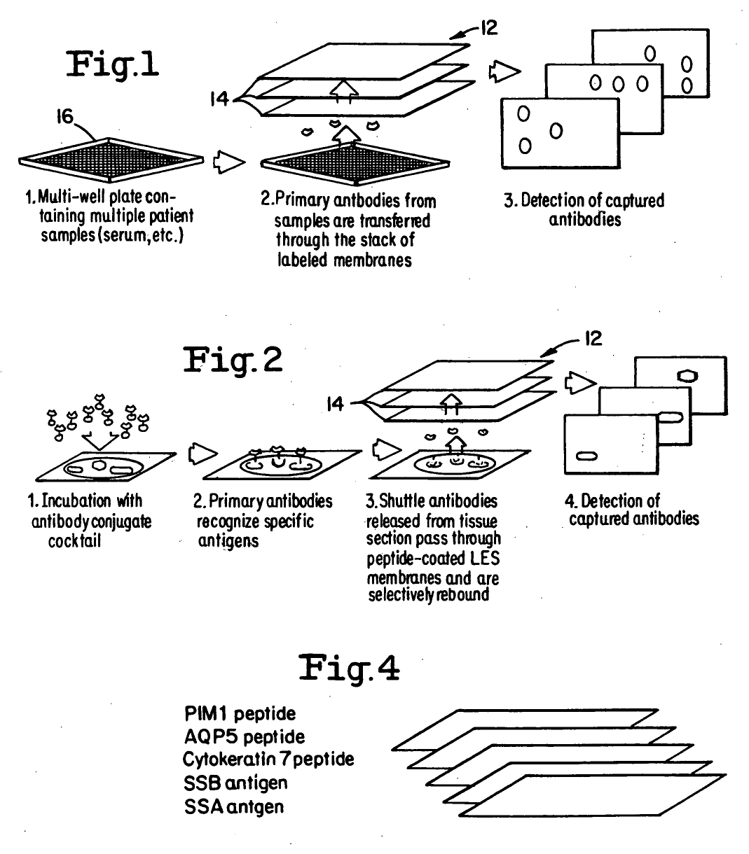

This method of the core technology, called indirect layered peptide array (iLPA), permits measurement of antibodies that are pre-bound to target antigens on a tissue section or immunoblot. The iLPA application is diagrammed in FIG. 2. Antibodies are hybridized to target antigens on a solid surface such as a tissue section or a standard immunoblot. The antibodies are then released from their antigens, passed through the membrane layers, and each antibody is measured on the appropriate peptide-coated membrane. In other words, the antibodies serve as reporters for the amount of antigen present in the specimen under study. Since the two-dimensional architecture of the specimen is maintained, all of the sub-elements in the samples are simultaneously measured, for example different histological regions of a tissue section, or protein bands on a blot.

The biomolecular content of the sample may elute directly into the membrane stack. Alternatively, in reference to FIG. 2 a cocktail consisting of conjugated antibodies may be applied to the sample, which ideally is a histopathological sample (or antigen in serum or other bodily fluids). These conjugated antibodies may be two antibodies: a primary antibody, and a shuttle antibody. The primary antibody is selected to recognize specific content in the sample. The shuttle antibody recognizes the peptide that coats the membranes that in turn corresponds to the peptide recognized by the primary antibody. The different shuttle antibodies will bind to different membrane layers based on the characteristics of the shuttle antibody. The shuttle antibodies are tested for selective binding in such a stack before conjugation with primary antibodies. Only one specific primary antibody is conjugated with one specific shuttle antibody, and vice versa. It should be noted that the shuttle antibodies need to be disattached from the primary antibodies in order to travel and bind to their corresponding peptides while maintaining the 2D structure.

Biomolecules detected on the membrane copies may be correlated with a biological characteristic of the sample. For example, a tissue specimen may be placed in a position on top of the stack, and a biomolecule of interest (such as a particular protein) may be detected in one of the membrane copies at a position that corresponds to the position in which the tissue specimen (or one of its substructures such as an organelle) was placed. The presence of that biomolecule in the tissue specimen can then be correlated with a biological characteristic of the sample. For example, a highly malignant tissue specimen may be found to contain a protein, which may then be associated with the highly malignant phenotype of the specimen.

Once the cocktail is applied to the sample, the primary antibody binds to the biomolecular content in the sample. The unreacted conjugated antibodies are then washed away, and the remaining conjugated antibodies are cleaved at the site of the primary and shuttle antibody's conjugation. The shuttle antibodies then elute through the membrane stack. After the elution, the membranes may be separated, and treated with a biomolecule that will detect the presence of the bound biomolecular content, so that it may be visualized on the membrane.

Alternatively, the invention can employ indirect capture to indicate the presence of the biomolecule of interest. In this method, a bispecific antibody conjugate cocktail is incubated with the sample. A bispecific antibody conjugate is a molecule consisting of two conjugated antibodies. The two conjugated antibodies are a primary antibody, qualified for immunohistochemistry, and a shuttle antibody, qualified for immunoblotting. The primary antibody recognizes a specific biomolecule in the sample. After being cleaved from the primary antibody, the shuttle antibody elutes through the membrane stack, selectively bonding to specific membranes.

The biological sample for direct capture may be serum, saliva, or other liquids. In particular embodiments, a sample may be present in each well of a multi-well plate, and biomolecules will elute through a stack of membranes, each membrane of which is coated to detect a specific molecule. The biological sample for indirect capture may be tissue.

The following are several non-limiting examples of uses and applications of the present invention.

### EXAMPLES

### Example 1

### Layered Membrane Capture of Antibodies from Serum of Patients with Sjogren's Syndrome

In this example, the ability of a layered peptide array (LPA) platform to detect and quantify antibodies was evaluated. Throughput capability, sensitivity, and specificity of the assay were evaluated using purified antibodies or antibody combinations under a variety of experimental conditions. To evaluate its clinical effectiveness, serum samples from Sjögren's syndrome (SS) patients, an autoimmune connective tissue disorder with characteristic auto-antibodies (4), were analyzed and the data compared to that derived from matching enzyme linked immunoabsorbent assays (ELISAs).

Antibodies and Serum Samples

Serum samples were collected from 35 Sjögren's syndrome patients who were diagnosed at the National Institutes of Health (NIH) Salivary Gland Dysfunction Clinic. Similarly, serum was extracted from eight healthy volunteers. All individuals signed consent forms to participate in a clinical research study that was approved by the IRB (study number 84-D-0056 and 94-D-0018). Serum was tested, on the day of collection at the NIH clinical center, for the presence or absence of anti-SSA and anti-SSB as determined by ELISA (Hemagen Diagnostics, Columbia, Md., USA). Antibodies and peptides used in the study are shown in table I. All dilutions were performed in phosphate buffered saline, pH 7.4 (Invitrogen corporation, MD, USA). Detection of antibodies on membranes was done using secondary rabbit anti goat-Fluoresceinisothiocyanate (FITC), goat anti human IgG-FITC or mouse anti rabbit-FITC in a dilution of 1:400 (catalog numbers sc-2777, sc-2456, sc-2359 accordingly, Santa Cruz Tech. CA, USA).

TABLE I
Antibodies and Antigens

| Antibody | Antigen | Catalog number | Company | Dilutions |
|---|---|---|---|---|
| Cytokeratin 7 | | sc-17116 | Santa Cruz tech. CA, USA | 1:50(4 ng/μl), 1:100 |
| Goat anti human | | | | 1:200, 1:400 |
| | Cytokeratin 7 peptide | sc-17116p | Santa Cruz tech. CA, USA | 2 μg/ml |
| AQP5 | | sc-9890 | Santa Cruz tech. CA, USA | 1:50(4 ng/μl), 1:100 |
| Goat anti human | | | | 1:200, 1:400 |
| | AQP5 peptide | sc-9890p | Santa Cruz tech. CA, USA | 2 μg/ml |
| Pim-1 | | sc-7856 | Santa Cruz tech. CA, USA | 1:50(4 ng/μl), 1:100 |
| Goat anti human | | | | 1:200, 1:400 |
| | Pim-1 peptide | sc-7856p | Santa Cruz tech. CA, USA | 2 μg/ml |
| Muscarinic | | sc-7474 | Santa Cruz tech. CA, USA | 1:50(4 ng/μl), 1:100 |
| acetylcholine receptor | | | | 1:200, 1:400 |
| | Muscarinic | sc-7474p | Santa Cruz tech. CA, USA | 2 μg/ml |
| | acetylcholine receptor | | | |
| Lactoperoxidase | | RAB/LPO | Nordic | 1:1001:200, |
| Rabbit anti bovin | | | immunology, Tilburg, | 1:400, 1:600 |
| | Lactoperoxidase | L8257 | Sigma - Aldrich | 25 μg/ml |
| | antigen | | MO, USA | |
| FAS | | sc-715 | Santa Cruz tech. CA, USA | 1:50(4 ng/μl), 1:100 |
| rabbit anti human | | | | 1:200, 1:400 |
| | FAS | sc-715p | Santa Cruz tech. CA, USA | 2 μg/ml |
| | Peptide | | | |
| Helicobacter pylori | | sc-17450 | Santa Cruz tech. CA, USA | 1:50(4 ng/μl), 1:100 |
| (CagA) | | | | 1:200, 1:400 |
| | Helicobacter pylori | sc-17450p | Santa Cruz tech. CA, USA | 2 μg/ml |
| | (CagA) | | | |
| Chlamydia, MOMP | | sc-17376 | Santa Cruz tech. CA, USA | 1:50(4 ng/μl), 1:100 |
| Goat anti | | | | 1:200, 1:400 |
| | Chlamydia, MOMP | sc-17376p | Santa Cruz tech. CA, USA | 2 μg/ml |
| | Peptide | | | |
| Caspase 3 | | sc-1225 | Santa Cruz tech. CA, USA | 1:50(4 ng/μl), 1:100 |
| Goat anti human | | | | 1:200, 1:400 |
| | Caspase 3 | sc-1225p | Santa Cruz tech. CA, USA | 2 μg/ml |
| | Peptide | | | |
| Coxsackie and | | sc-10314 | Santa Cruz tech. CA, USA | 1:50(4 ng/μl), 1:100 |
| adenovirus receptor | | | | 1:200, 1:400 |
| | Caspase 3 | sc-10314p | Santa Cruz tech. CA, USA | 2 μg/ml |
| | Peptide | | | |
| Human autoantibody | | HSA-0100 | Immunovision, AR, USA | 1:200 |
| against SSA antigen | | | | |
| | SSA antigen | SSA-3000 | Immunovision, AR, USA | 7.5 μg/ml |
| Human autoantibody | | HSB-0100 | Immunovision, AR, USA | 1:200 |
| against SSB antigen | | | | |
| | SSB antigen | SSB-3000 | Immunovision, AR, USA | 7.8 μg/ml |

Enzyme-Linked Immunosorbent Assay (ELISA)

Serum samples were evaluated for SSB using an ELISA kit (Hemagen Diagnostics, Columbia, Md., USA) according to the manufacturers' recommendation.

### Layered Peptide Array Coated Membranes

Poretics® Polycarbonate PVP Free Membranes (0.4 micron) from GE Osmonics (Minnetonka, Minn.) were used in these examples. The membranes were coated according to the following process:

Membranes were incubated in a solution that contained peptides (table I) or the antigen (table I) diluted in PBS. Every membrane was incubated with only one peptide, though two peptides (or a peptide and antigen) can coat one membrane and successfully bind two antibodies that are specific for that membrane. The membranes were incubated with the peptides/antigens for four hours at room temperature with shaking. Membranes were washed in a TBST solution ((1) IM Tris HCl pH 8.0-50 ml (50 mM); (2) 5 M NaCl-ml (150 mM); (3) Tween 20 0.5 ml (150 mM), in a total volume of 1 liter ddH₂O), three times for 5 minutes and applied to the LPA system. The membranes were cut to an appropriate size to fit the dimensions of the gel or the 96 well plate employed.

### Layered Peptide Array—Prototype 1

Membranes were equilibrated in transfer buffer (TB) (50 mM Tris, 6.07 gr. 380 mM Glycine, 28.54 g in 1 liter of deonized water). A 2% agarose gel (Gibco BRL, NY, USA) was prepared according to the manufacturers' recommendation in a B-1 casting booth (OWL separation systems, NH, USA), and then a 14-well comb was inserted.

The wells were loaded with antibodies or serum samples diluted in PBS as shown in Table 1. The gel was supported on a gel blot paper GB004 (Schleicher & Schuell, NH, USA) and a multilayered system for capillary transfer was prepared as follows (from lower to upper layers):

a. Large tray (14×20 cm)
b. Inverted B1 casting booth
c. Gel supported by blot paper
d. LPA coated membranes
e. 20 blot papers (5×8 cm)
f. Weight (7-10 gram per cm²)
g. Sealing wrap

The inverted B1 casting booth was put in a large-tray. The gel, supported by the blot paper, was put in the inverted casting booth. The antibody affinity membranes was put on top of the gel. Blot papers were placed on top of the membranes, whereupon the weight is placed on top of the blotting papers. The entire complex was surrounded by saran wrap to prevent moisture loss.

Transfers were done overnight, followed by washing of membranes three times in TBST (50 mM Tris Hcl, 150 mM NaCl, 150 mM Tween20) for five minutes each, and then incubation with 2^{nd} antibody for 30 minutes at room temperature with shaking, followed by another wash in TBST. Membranes were dried on a filter paper (Whatman, N.J., USA) and scanned on a Typhoon scanner with 520 BP40 filter (Typhoon 9410, Amersham Biosciences, NJ, USA).

### Layered Peptide Array—Prototype #2

LPA affinity membranes were placed within a vacuum plate (Bio-Rad, CA, USA). Antibodies were applied to the 96 wells in the plate and incubated for 5 minutes. Vacuum was applied for 5 minutes followed by washing of the membranes in TBST, application of secondary antibodies, and scanning as described above.

Thirty-two SS serum or saliva, and 8 NV serum or saliva samples were placed in duplicates in the 96 well plate and P-FILM™ membranes coated with SSA, SSB, MOMP, CAR, CagA, M3, Fas and caspase 3 were placed in duplicates between the samples and the vacuum. 1 μl serum or 5 μl saliva were used per experiment for every patient/NV. The vacuum was performed for 5 minutes followed by disassembling the plate, washing the membranes, and reacting with secondary, fluorescein conjugated antibody following the protocol for prototype I. Each experiment was repeated 4 times and the mean membrane signal intensity was calculated for 8 total membranes for each peptide/antigen (4 experiments, two membranes per experiment for every antibody/peptide set) for each patient.

Normalization with a lactoperoxidase antibody-antigen pair was performed in each well. The patient and NV samples were placed in an arbitrary order in the plate and the calculation of the signal was done in a blinded manner. Unmasking of the groups was done after the signals were averaged and the standard deviation calculated. Background activity was calculated as the signal of the non relevant positive control antibodies for the other membranes in the stack. For example: for the SSA and SSB membranes the background was the mean signal from the positive control antibodies for CAR, M3, caspase 3, caspase 1, AQP5, cytokeratin, 7 PIM1 in the same experiment.

### Data Analysis

### 1. Density Measurements

Images of the membranes were imported to the ImagePro 4.5 analysis software (MediaCybernetics, MD, USA) for analysis. Each membrane included slots according to the amount of wells (in prototype 1) or 96 dots (in prototype 2). The optical density was calculated by the program for each well in the membrane by marking a rectangle around each slot (in prototype 1) or a circle containing each dot (in prototype 2). The optical density was defined according to the following formula:

[OD=−Log₁₀(×/256)]

With 256 representing the total number of gray levels in the image and X the individual level of gray of each object (each slot of the total slots per membrane in prototype 1 or each well of the 96 wells for each membrane in prototype 2).

The measurements were repeated 4 times. The position of the different coated membranes in the stack were varied each time. Thus, a data set of average optical densities was generated for each well in all the membranes. The data were imported to Microsoft Excel and mean a standard deviation values were calculated.

### 2 Comparison of LPA to ELISA Results

In order to compare the results of the ELISA, that is represented in arbitrary units, and the LPA values, that are represented in intensity of signal, both sets had to be normalized to transfer them to one common set of values between 0 and 1. Thus, each set was divided by the maximal result in this set.

### 3 Statistical Analysis Software

One-way analysis of variance (ANOVA) was applied to the data using PartekPro (Partek inc. St. Charles, Mo.). The Principal component Analysis (PCA) module of the Partek Pro software package (Partek inc. St. Charles, Mo.) was used to analyze the results: The data were imported from MicroSoft Excel to PartekPro 5.1 and a PCA scatterplot graph was generated for, the two different groups (SS patients and NV) with 520 measurements (mean values of 40 serum samples multiplied by 8 antibody groups=320, plus mean values of 40 saliva samples multiplied by 5 antibodies=200).

### Results

In the present study, measurement of 96 samples across 50 membranes, producing 4800 measurements in each experiment was demonstrated.

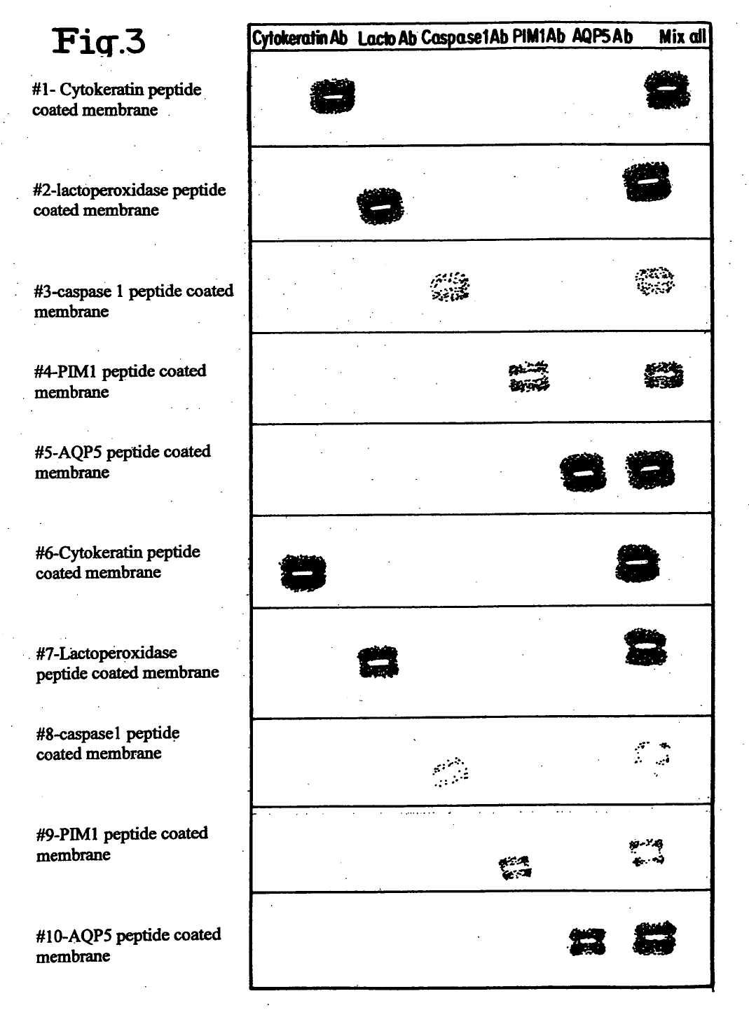

The initial evaluations of the LPA platform for antibody detection were performed using a first prototype system containing 10 layers and five different antibodies [against cytokeratin, lactoperoxidase, caspasel, PIM1, AQP5 (Aquaporin 5)]. To test reproducibility, peptides corresponding to each antibody were coated onto two different membranes within the stack (z-dimension); for example, cytokeratin peptide was coated onto membranes #1 and #6, lactoperoxidase peptide was coated onto membranes #2 and #7, and so on. The sample set (x-y plane) was comprised of six samples in individual wells, including each antibody in purified form (wells 1-5, respectively), and a sixth well that contained a mixture of the five antibodies together. The samples were passed slowly through the membrane stack overnight by capillary transfer, and the antibody capture was assessed on day two using a FITC-labeled secondary antibody. The results shown in FIG. 3 indicate that each antibody was captured on its corresponding peptide-coated membrane, with little or no non-specific background signal. Additionally, the capture occurred efficiently for single antibodies as well as with the antibody mixture.

Assays using clinical samples were evaluated next. In these experiments, the levels of two characteristic auto-antibodies used in an established classification criteria set for the auto-immune disease Sjögren's Syndrome (SS) were measured The assay format and experimental conditions were similar to those used in the experiment described above, except that proteins corresponding to Sjögren's Syndrome antigen A and antigen B (SSA and SSB, respectively) were used in place of the lactoperoxidase and caspasel peptides. FIG. 4 shows the order of the coatings among the five membranes (z-dimension) and FIG. 5 shows the data in bar graph form. The samples included: serum from three SS patients seen in a clinical study at the NIH known to be positive for SSA and/or SSB; positive control sera for SSA and SSB; normal human serum as a negative control; and purified antibodies corresponding to each of the five antigens coatings. The data shown in FIG. 5, indicate that the assay system was capable of reliably detecting the SSA and SSB antibodies in the clinical serum samples, similar to the corresponding purified antibodies. Serum from patient 1 was positive for SSA antibody only. Antibodies against SSB, cytokeratin, AQP5 and PIM1 were not detectable. Serum from patient 2 was positive for SSA, and slightly positive for SSB. Serum from patient 3 was positive for SSA and SSB. The positive and negative control samples performed as expected. The figures represents a summary of four different experiments with each sample run in triplicate.

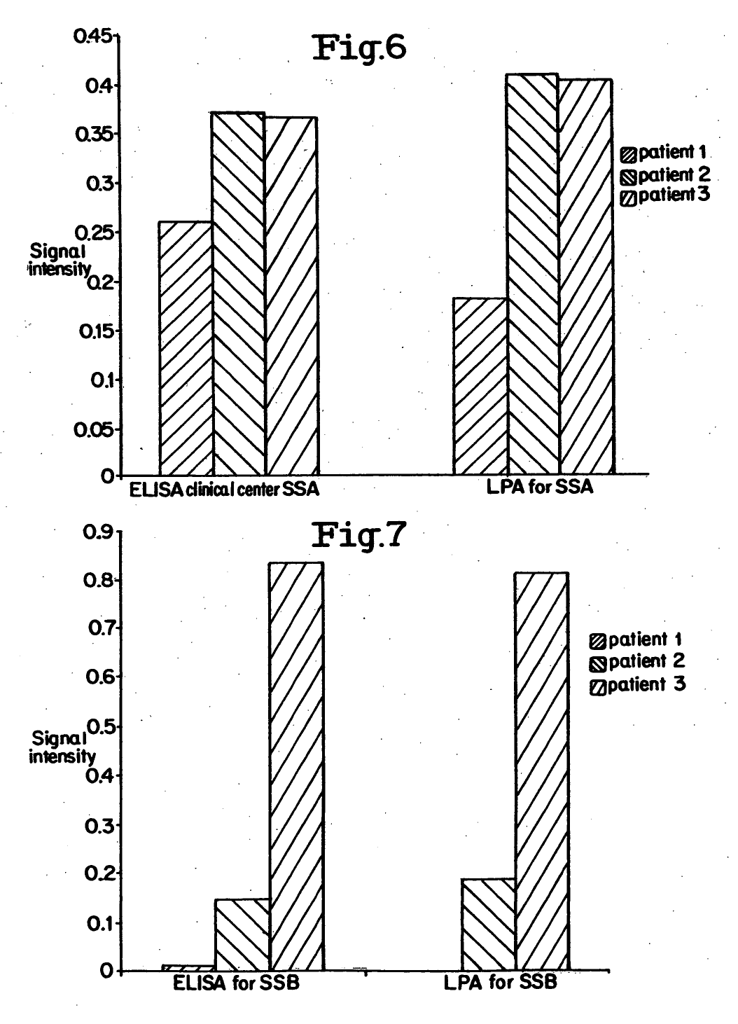

To further evaluate the data, the LPA results were compared to those derived from standard ELISAs performed at the NIH Clinical Center for the three SS patients. FIG. 6 shows all three were positive for SSA, with patients #2 and #3 showing SSA titers higher than patient #1. Similar results were seen on the LPA platform, as shown in the right side of FIG. 7. For SSB antigen, an ELISA was performed in the laboratory side-by-side with the LPA analysis. As seen in FIG. 7, the two approaches produced similar results, with patient #1 being negative for SSB by both methods, patient #2 positive for SSB, and patient #3 strongly positive for SSB.

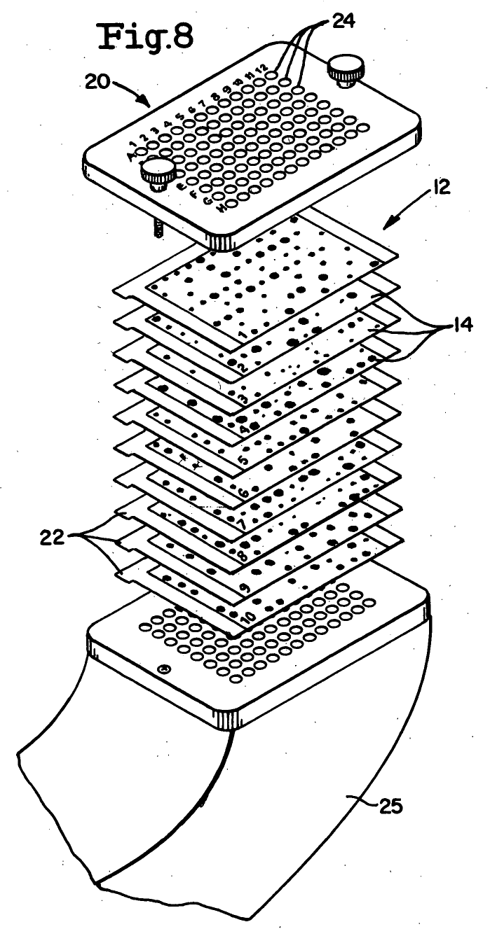

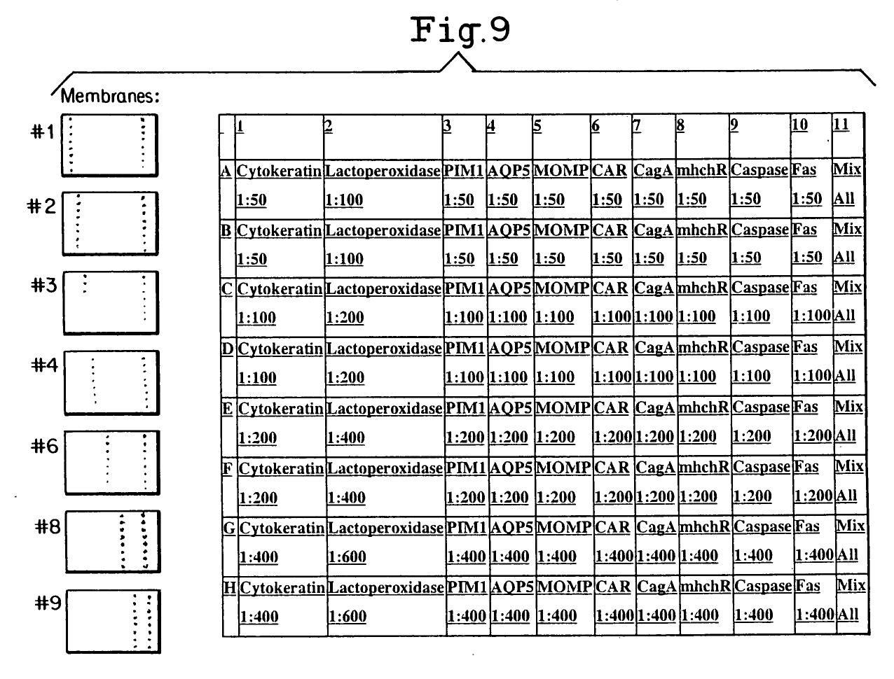

Having established the basic experimental parameters for the assay, an examination was made of a “second generation”-prototype system comprised of a 96-well vacuum plate and 50 membranes, capable of producing 4800 measurements per experiment (FIG. 8). The system was tested using purified antibodies as shown in the dilution chart of FIG. 9. The experiment utilized 10 antibody-antigen pairs, with each peptide coated on every tenth membrane, to produce five “identical” experiments. For example, cytokeratin peptide was coated on membranes #1, 11, 21, 31, 41, lactoperoxidase peptide was coated on membranes #2, 12, 22, 32, 42, and so on. The vacuum-based prototype system offered the advantage of well-controlled movement of the samples through the membrane stack; hence, transit times were examined. It was determined that antibody capture occurred efficiently in as short as five minutes. The 96-well grid contained each of the antibodies at four separate dilutions, as well as a mixture of the antibodies together. The layout of FIG. 9 illustrates the layout of the x-y grid along with the first set of 7 membranes that showed a positive signal (z axis). Membrane #1 was coated with cytokeratin peptide, membrane #2-lactoperoxidase antigen, membrane #3-PIM1 peptide, membrane #4-AQP5 peptide, membrane #6-CAR peptide, membrane #8-M3 peptide, and membrane #9-caspase 3 peptide.

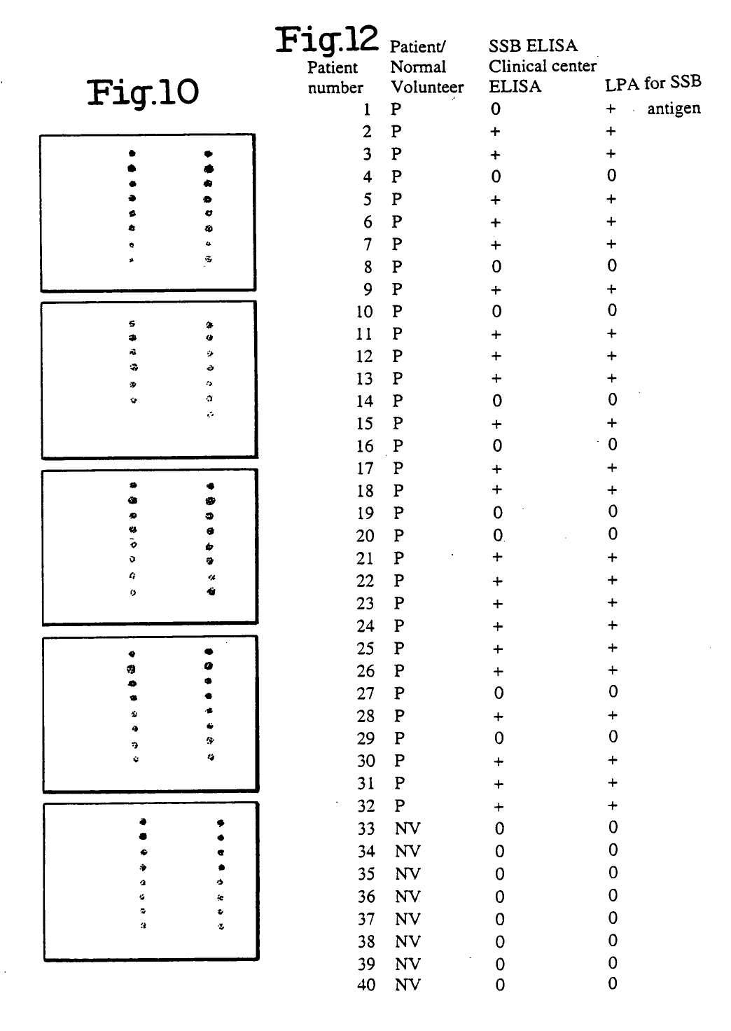

The positive results were highly reproducible among the five replicate membranes for each antibody-peptide/antigen, and little or no cross-reaction was observed among the antibodies and non-target membranes. This is illustrated in FIG. 10 showing capture of the CAR antibody by its peptide on membranes #6, 16, 26, 36, 46. Three antibody-peptide pairs (MOMP, CagA, FAS) were completely negative by LPA, and, as with the other experiments, the samples did not efficiently hybridize to peptides immobilized on a surface (data not shown). A simple rule of thumb for LPAs is that any commercially available antibody (or antibody in a patient sample) that recognizes its antigen on a blot (immunoblot, dot-blot) will work effectively in the system. FIG. 11 shows a dilution curve for each of the positive antibody-antigen pairs, indicating that LPAs are capable of measuring two-fold changes in antibody titer.

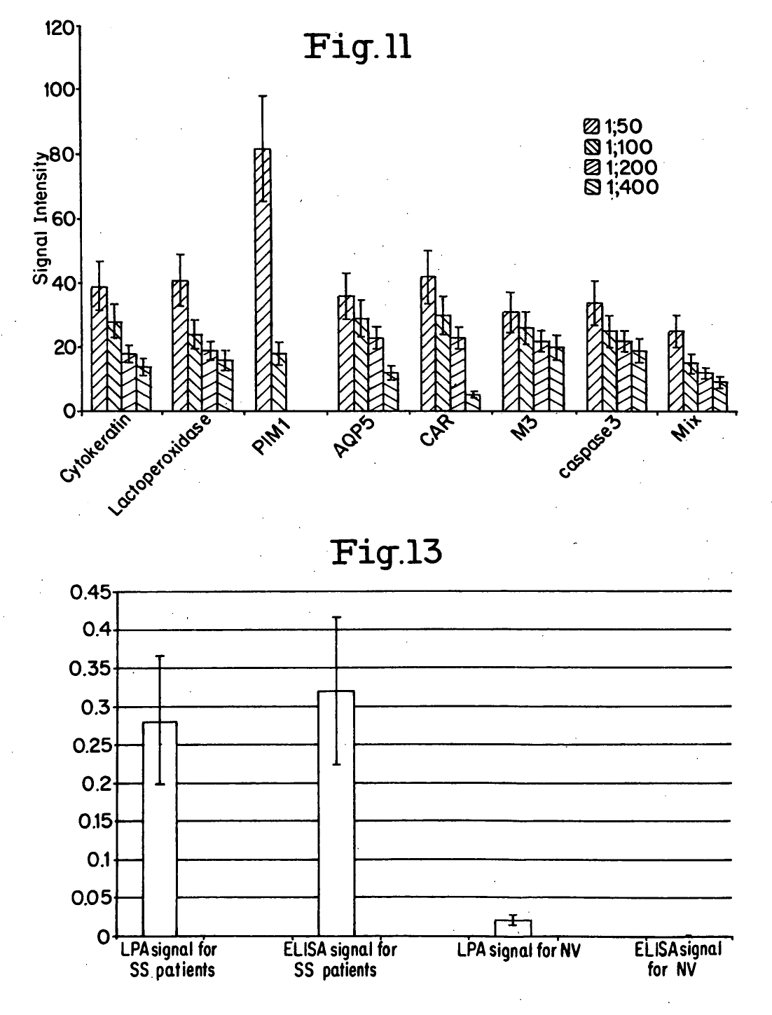

The next step in examining LPA prototype #2 was to analyze serum samples from the clinic. Thirty-two sera from SS patients and eight normal volunteer controls were assayed using 10 peptide-coated membranes. One of the membranes was coated with SSB antigen and the results from the LPA system correlated with standard ELISA measurements performed previously. The results are shown in panel A of FIG. 12. LPA analysis showed a sensitivity of 100% (correctly identified 22 out of 22 positive cases) compared to ELISA, and an LPA specificity of 94% (correctly identified 17 out of 18 negative cases). To investigate the single patient where a discrepancy was observed, a new ELISA test of this sample was performed. The assay showed that this symptomatic SS patient classified as positive by LPA indeed had low levels of SSB antigen as determined by ELISA, although it was below the Clinical Center threshold for assignment as a positive result. FIG. 13 shows the average signal for the SS patients and normal volunteers for both ELISA and LPA analysis. In each assay system, the average intra-patient reproducibility was similar. There was a standard deviation of 0.1 for LPA, and a standard deviation of 0.14 for ELISA.

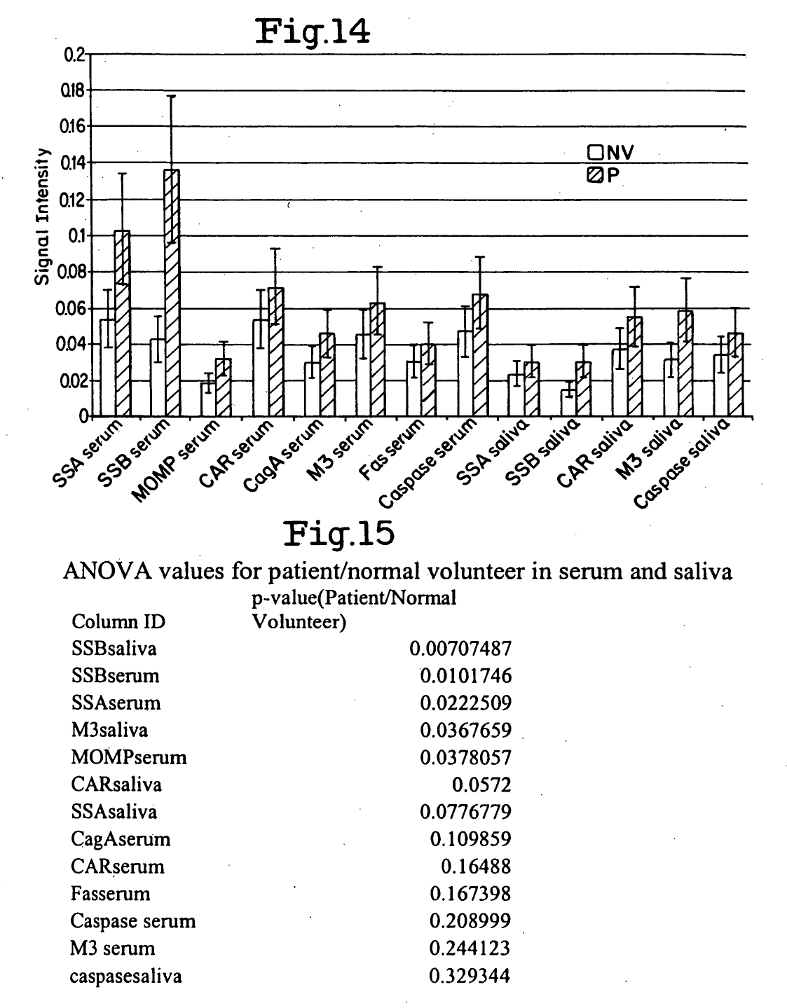

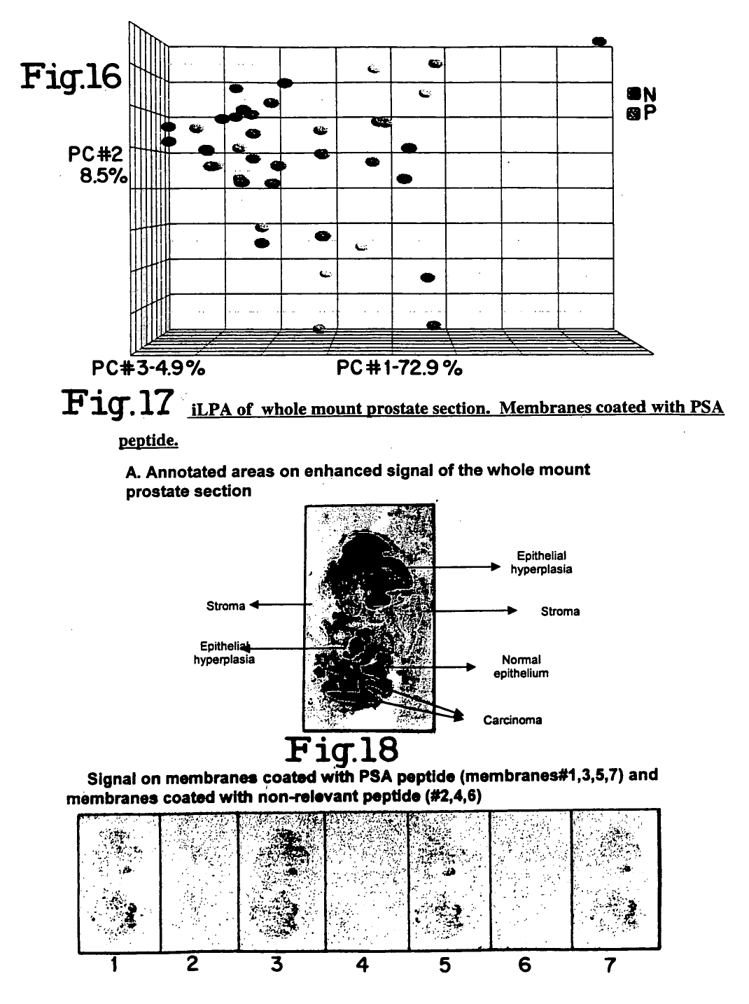

Next, the LPA platform was used to analyze 80 clinical samples for SSA and SSB, as well as for several auto-antibodies previously detected in autoimmune disorders. In these experiments, the test set included serum samples from 32 SS patients and eight healthy control subjects, and saliva samples from the same 32 SS patients, and control group. The analysis membranes were coated with the following peptides: SSA, SSB, MOMP, CAR, CagA, M3, Fas, and caspase 3 as shown in FIG. 14. Each sample was run in duplicate and repeated four times. As expected, SSA and SSB were statistically elevated in the patient's serum as compared to controls. The other auto-immune related antibodies were also elevated in the patients, although the increase was less pronounced and was statistically significant for only two of the comparisons (FIG. 15). The multiplex capability of the LPA platform enabled the determination of the overall difference in serum and saliva immunoreactivity between the patient and control groups. By combining the data together, the SS patients and normal volunteers segregate from each other at a p value of 0.0000427 for serum and 0.000798544 in saliva. This effect is shown graphically in FIG. 16 using principal component analysis (PCA) clustering. Moreover, the multiplex data allowed for an examination of a relationship between the titer of each antibody, or various antibody combinations, and the pathology of the patients. No statistically significant correlation was observed among the early, moderate, or advanced disease categories (data not shown).

### Example 2

### Layered Membrane Capture of Antibodies from Tissues of Patients with Sjogren's Syndrome

Layered Membrane Capture of “Shuttle” Antibodies from Tissue Section

To evaluate the iLPA approach, patient tissue samples from prostate cancer and Sjorgrens syndrome patients were studied and the data compared to that derived using standard immunohistochemical analysis. Quantitative, multiplex proteomic analysis of histological sections was achieved, with sensitivity and specificity similar to standard immunohistochemistry. Overall, the experiments using iLPA technology suggest the method will be a simple, versatile, and relatively inexpensive method for multiplex molecular measurements from biological samples.

### Materials and Methods

### Tissue Samples

Prostectomy—cases were obtained from the National Institutes of Health and the National Naval Medical Center under an IRB-approved protocol. Whole-mount prostate cancer cases were ethanol-fixed and paraffin-embedded. Tissue sections were cut to 10 μm thickness for the iLPA protocol.

Minor Salivary Gland Tissues-1

Labial minor salivary gland tissues were obtained from nine patients with primary SS and two healthy volunteers. All patients met the revised EC criteria for diagnosis of primary SS, and none had another connective tissue disease. Healthy volunteers had no complaints of oral or ocular dryness, no autoimmune serologies, and normal salivary function as assessed by salivary flow rates. All human tissue samples were acquired and utilized in accordance with approvals from the NIDCR human subject review committee. Immediately after removal, specimens were placed in OCT compound, snap frozen in methyl butane on dry ice, held overnight at −70° C., and then stored in liquid nitrogen until use. Tissue samples were cut at a 10 μm thickness for the iLPA protocol. Each section was placed on a charged glass slide.

### Layered Expression Scanning Coated Membranes

LPA affinity membranes were used in the study (commercially available exclusively from 20/20 GeneSystems, Inc., Rockville, Md., www.2020gene.com).

iLPA Protocol

Ethanol fixed prostate tissue was placed in 60° C. oven for 1 hour followed by 2 washes with xylene. Rehydration with alcohol was performed using 100, 95 and 70% alcohol. Sections were blocked for 10 minutes with blocking serum and incubated for 1 hour at RT with a cocktail of the following primary antibodies: cytokeratin 7, PSA and CD4 antibodies. After washing: the sections, the coated membranes were stacked on the tissue, soaked in transfer buffer, and transfer pads were placed on top of the stack. The stack was placed in a plastic pocket, sealed, and placed on a hot plate at 37° C. for 1 hour, and 45° C. for the second hour. The stack was disassembled and the membranes were reacted with a secondary anti-goat antibody FITC conjugated and analyzed with a Typhoon scanner using a fluorescence filter (FITC-absorption-490 nm, emission 520 nm).

Frozen minor salivary gland tissues were fixed for 10 minutes in ice-cold acetone and a similar protocol for iLPA was performed. The primary antibody cocktail applied on the tissues was: Cytokeratin 5, AQP5, M3 and caspase 3 antibodies.

### Analysis of Results

Prostate tissue membranes were analyzed and qualitatively correlated with standard immunohistochemical analysis performed on an adjacent recut section. Minor salivary gland membranes were quantified using an ImagePro image analysis program for signal intensity. A mean was obtained for all cases: in every case two tissues were present on every section in every experiment. Every experiment had 2 repeats of the same peptide coating in the stack and the experiments were repeated 4 times, with varying the locations of the peptide coated membranes in the stack (For example: cytokeratin 7 peptide was coated on membrane #1 and #5 in experiment 1, on membranes #2 and #6 in experiment 2, on membranes #3 and #7 in experiment 3 and on membranes #4 and #8 in experiment 4). Thus every patient had 16 values for every antibody-peptide pair.

### Results

The iLPA approach was evaluated using two tissue types. Initially, whole-mount prostate tissue sections were studied so that both the measurement capability and the histological resolution of the system could be assessed (FIG. 17). The tissue section was pre-incubated with Prostate specific antibody (PSA) antibody similar to standard immunohistochemical analysis. The section was placed adjacent to seven analysis membranes. Membranes number 1, 3, 5 and 7 were coated with PSA peptide and membranes 2, 4, 6 were coated with irrelevant peptide; the antibodies were released from the tissue, and subsequently captured on their respective peptide-coated membrane. The signal for PSA was annotated according to immunohistochemistry of PSA antibody on section from a consecutive cut and a very close correlation was achieved. The signal was completely missing on the non relevant P-Film coated membranes and the prostate tissue showed a very consistent pattern of expression on the P-Film PSA coated membranes according to the epithelium areas (FIG. 18).

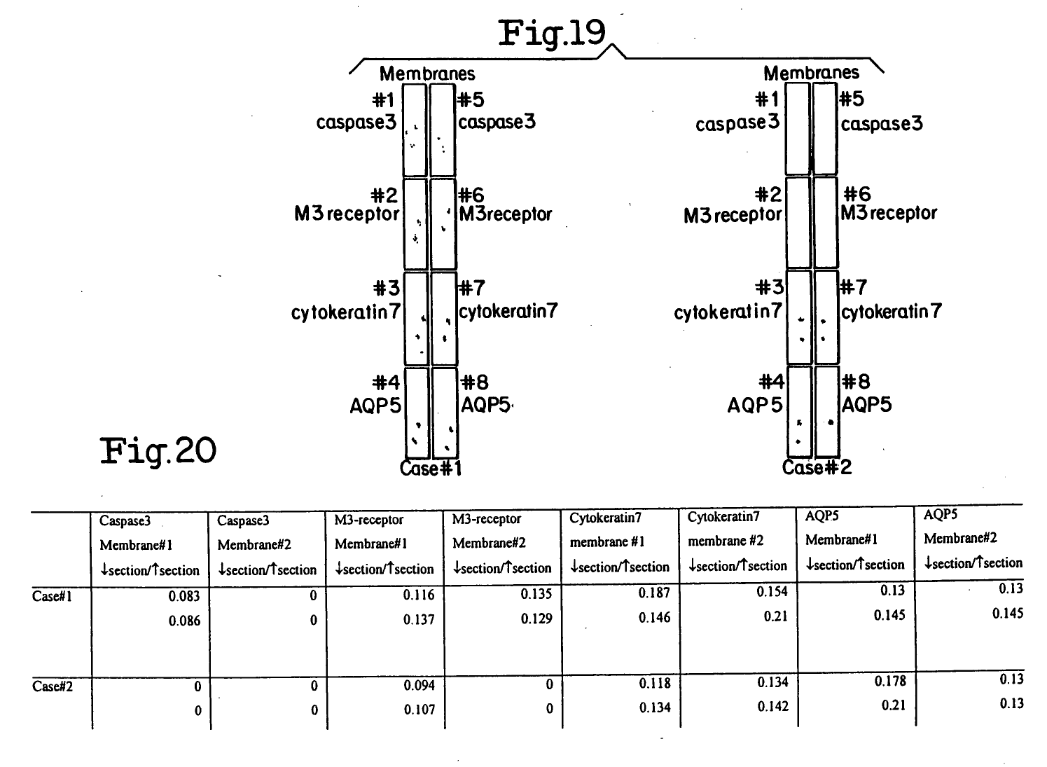

FIGS. 19 and 20 represent results from minor salivary gland tissues. Salivary gland tissue showed strong signal for cytokeratin 7 and AQP5 moderate signal for M3 receptor and almost no signal for caspase 3 antibodies. These results are in line with publications of signal intensity for these antibodies in minor glands in Sjögren's syndrome.

FIG. 24 is a representative example of 2 cases for minor salivary gland signal on 8 membranes. Every case was repeated 4 times and signal was obtained for every antibody-peptide pair two times in every experiment. In this figure the intensity levels were according to the table and very closely related.

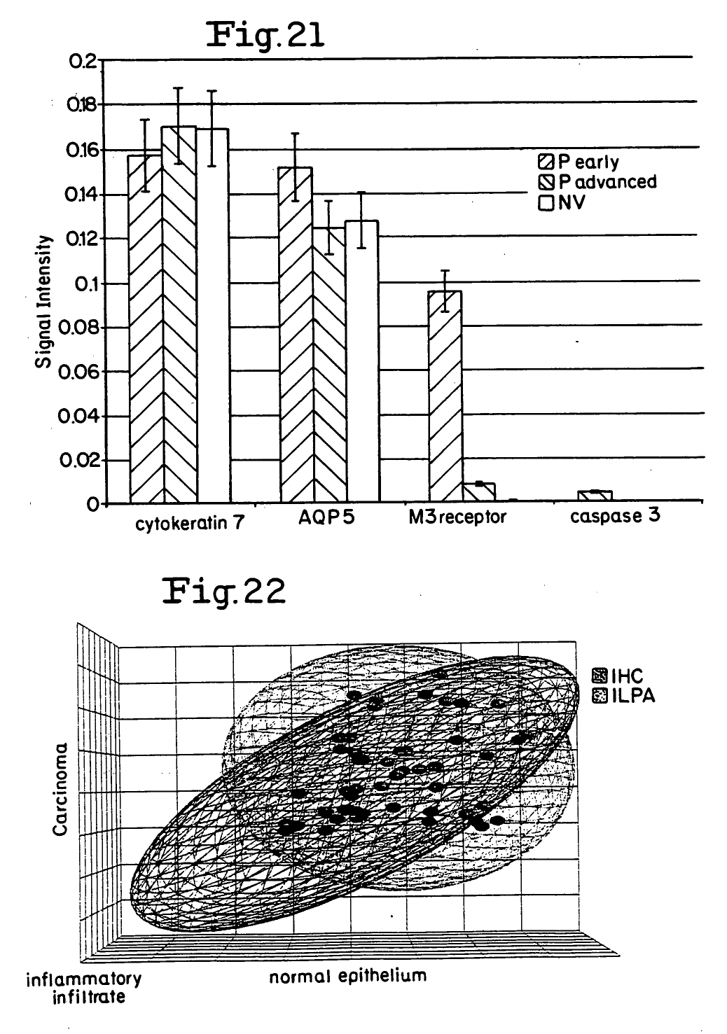

FIG. 21 represents the mean measurements for all 11 minor gland tissues grouped according to early disease, advanced and normal volunteers. The standard deviation values for all the measurements were very small.

### Results

The iLPA approach was evaluated using two tissue types. Initially, whole-mount prostate tissue sections were studied so that both the measurement capability and the histological resolution of the system could be assessed (FIG. 19). The tissue section was pre-incubated with prostate specific antibody (PSA) antibody similar to standard immunohistochemical analysis. The section was placed adjacent to seven analysis membranes. Membranes 1, 3, 5, and 7 were coated with PSA peptide and membranes 2, 4, and 6 were coated with an irrelevant peptide. The antibodies were released from the tissue, passed through the analysis layers, and subsequently captured on their respective PSA peptide-coated membrane. The prostate tissue showed a very consistent pattern of expression on the LPA membranes matching the epithelial areas (FIG. 18), and similar to that of the recut section stained by standard immunohistochemistry. Signal was absent on the non relevant LPA coated membranes. As shown in FIG. 18, Panel B demonstrates the P-Films according to their sequence of application on the tissue. The P-Films that were coated with non relevant peptide were blank (membranes 2, 4, 6) whereas the PSA coated membranes showed a steady and reproducible signal on the membranes (membranes #1, 3, 5, 7)

FIGS. 19-21 are iLPA results from minor salivary gland tissues showing strong signal for cytokeratin 7 and AQP5, moderate signal for M3 receptor, and little or no signal for caspase 3 antibodies. More specifically, whole proteins were spotted on nitrocellulose coated slides. The slide on the left was spotted with a dilution curve (higher concentration upper lower concentration bottom) of a head and neck carcinoma cell line, HEP2. The middle slide was spotted with a dilution curve of lactoperoxidase protein and the slide on the right was spotted with a dilution curve of Albumin. The same cocktail of antibodies was applied to all slides consisting of cytokeratin 7; lactoperoxidase and albumin. After one hour of incubation the slides were washed and the contact transfer was carried out with membranes coated with cytokeratin, lactoperoxidase or albumin.

These results are consistent with what is known about Sjögren's syndrome. FIGS. 19 and 20 are representative examples of two cases of minor salivary gland shown on eight membranes. Whole mount prostate tissue was reacted with PSA antibody and P-Fils coated with PSA peptide and non-relevant peptide were applied to the tissue. The signal on P-Film coated with PSA peptide was annotated according to immunohistochemistry for PSA antibody. These results are illustrated in FIG. 21, which represents the mean measurements for all 11 minor gland tissues grouped according to early disease, advanced disease, and normal volunteers. The reproducibility of the iLPA technique was excellent and the standard deviation values for the measurements were small. FIG. 21 summarizes the results per case of Sjögren's syndrome patients and controls to show the iLPA system reproducibility. Every bar represents a summary of 16 measurements.

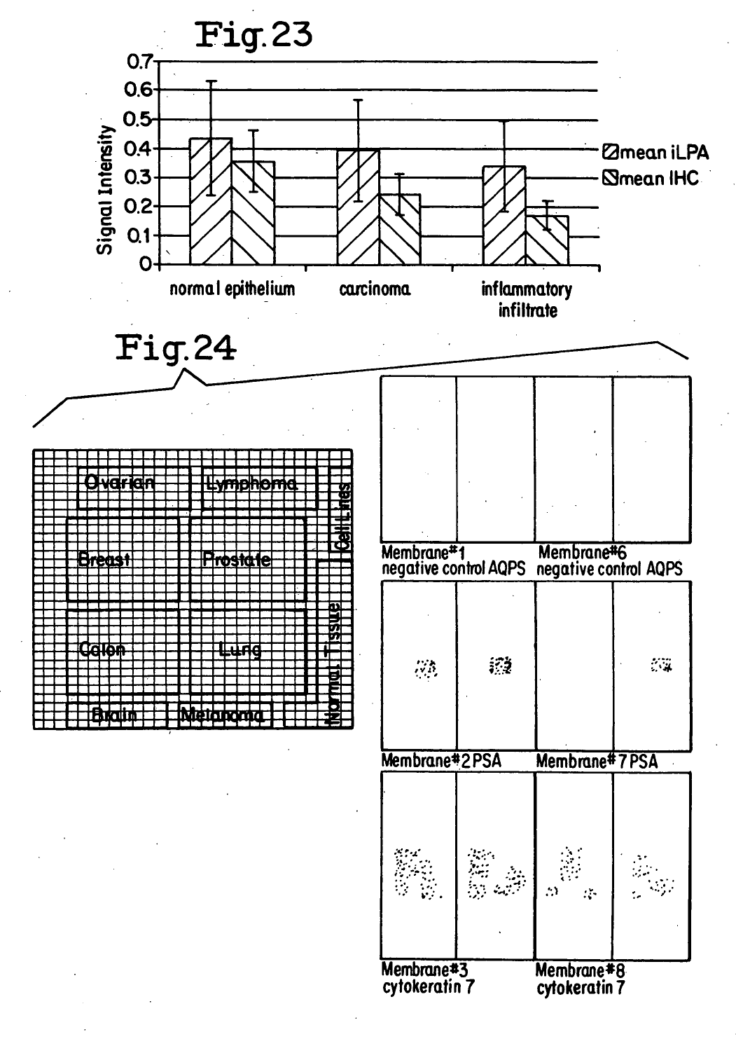

A summary for 11 minor salivary gland tissue sections (16 repeats for every tissue) divided according to early disease (Pearly), advanced disease (P advanced) and the normal volunteer tissue is found in FIG. 23 (NV). The iPla shows a greater intensity of the signal.

10 cases of prostate whole mount frozen prostate cases were used in this study. iLPA experiment was carried out on one section from every case including membranes coated with PSA, cytokeratin 7 and CD4 peptides. Tissue was reacted with a cocktail of cytokeratin7, PSA and CD4 antibodies and results were counted using an ImagePro program. Consecutive sections from every case were reacted immunohistochemically with either CD4, cytokeratin 7 or PSA. The data was quantitatively measured using ImagePro program for three areas: normal epithelium, carcinoma and inflammatory infiltrate. FIG. 22 shows that IHC and iLPA values are within the same area count when using the PCA clustering by PartekPro and FIG. 8 shows that the two methods have the same trend of values using the bar graph.

Additionally, two formalin fixed and paraffin embedded tissue arrays from TARP1 generation were used. Referring to FIG. 24, the different cancers on the arrays are organized according to the chart on the left. The slides were deparafinized and the slide on the right was treated for antigen retrieval the slide on the left was not pretreated. Both slides were reacted with antibodies for PSA and sytokeratin and iLPA system was applied using contact transfer and membranes coated with AQP5 (negative control) on membranes 1 and 6 along with PSA (on membranes 2 and 7) and cytokeratin (membranes 3 and 8). The figure shows that the tissues positive for PSA are only the prostate cancer-whereas the tissues positive for cytokeratin 7 were ovarian, breast, prostate colon and lung, but not brain and lymphomas.

Many modifications and variations of the present invention are possible in light of the above teachings. It is, therefore, to be understood within the scope of the appended claims the invention may be protected otherwise than as specifically described.

1. (canceled)

2. The method of claim 24, wherein said biomolecule is an antibody.

3. (canceled)

4. (canceled)

5. (canceled)

6. (canceled)

7. (canceled)

8. (canceled)

9. (canceled)

10. The method of claim 1 wherein infectious agents are identified.

11. The method of claim 1, wherein toxins are identified.

12. The method of claim 1, wherein more than one sample may be tested per test run.

13. (canceled)

14. The method of claim 21, wherein said physiological event is selected from the group consisting of autoimmune diseases, pathological infections, and toxic events.

15. (canceled)

16. The method of claim of claim 21, wherein said infectious pathogens are selected from the group consisting of bacteria, viruses, molds, and funguses.

17. The method of claim 17, wherein said toxic events are selected from the group of bacterial toxins and environmental toxins.

18. (canceled)

19. (canceled)

20. (canceled)

21. A method for identifying a histopathological tissue sample, said method comprising:
a) obtaining said histopathological sample;
b) exposing said histopathological sample to conjugate antibodies, said conjugate antibodies comprising:
i) a primary antibody, said primary antibody having specificity for certain specific peptides of a sample;
ii) a shuttle antibody attached to said primary antibody, forming a bispecific antibody conjugate;
c) obtaining a coated capture membrane stack, said membrane stack comprising a plurality of coated capture membranes, each said coated capture membrane of said coated capture membrane stack comprising:
i) a membrane; and
ii) a peptide coating said membrane, wherein each said peptide coating of each said coated capture membrane differs from other said peptide coatings of said coated capture membranes of said coated capture membrane stack;
d) washing away any unreacted antibodies,
e) exposing said coated capture membrane stack to said histophathological sample for a given time period to allow for cleaving of conjugated antibodies at the site of the primary and the shuttle antibody conjugation, allowing for said shuttle antibodies to migrate to any said peptide coated membrane for which said shuttle antibody is specific;
f) separating each said coated capture membrane from said membrane stack; and
g) identifying said histological sample, by identifying at least each said coated capture membrane to which at least one said shuttle antibody adheres.

22. A kit for identifying antibodies, said kit comprising:
a membrane stack, said membrane stack comprising:
a) a plurality of capture membranes, each said capture membrane being a track etched membrane and;
b) each said capture membrane coated with a different peptide.

23. The method of claim 21, wherein a single species of primary antibody is conjugated with only one species of shuttle antibody.

24. A method for identifying antigens of a histopathological tissue sample, said method comprising:
a) obtaining said histopathological sample;
b) exposing said histopathological sample to antibodies which target specific antigens on a histopathological sample;
c) obtaining a coated capture membrane stack, said membrane stack comprising a plurality of coated capture membranes, each said coated capture membrane of said coated capture membrane stack comprising:
i) a membrane; and
ii) a peptide coating said membrane, wherein each said peptide coating of each said coated capture membrane differs from other said peptide coatings of said coated capture membranes of said coated capture membrane stack;
d) exposing said coated capture membrane stack to said histopathological sample for a given time period to allow said antibodies to migrate to any said peptide coated membrane for which said antibodies are specific; and
f) identifying said antigen of said histological sample, by identifying at least each said coated capture membrane to which said antibodies adhere.

25. The method of claim 24, wherein said coated capture membrane is a track etched membrane.

26. The method of claim 24, wherein said capture membranes are separated before identification of said antigen of said histological sample.
